(19) United States
(12) Patent Application Publication
(10) Pub. No.: US 2012/0046264 A1
(43) Pub. Date: Feb. 23, 2012

(54) COMMERCIAL SCALE PRODUCTION METHODS FOR TRANSDERMAL HORMONE FORMULATIONS

(75) Inventors: Stephen Simes, Long Grove, IL (US); Linda Lieb, Lake of the Hills, IL (US)

(73) Assignee: BIOSANTE PHARMACEUTICALS, INC., Lincolnshire, IL (US)

(21) Appl. No.: 13/212,051

(22) Filed: Aug. 17, 2011

Related U.S. Application Data
(60) Provisional application No. 61/401,640, filed on Aug. 17, 2010.

(51) Int. Cl. A61K 31/568; A61K 31/343; A61P 15/00; A61K 31/565; A61K 31/57; A61K 31/566; A61K 31/366

(52) U.S. Cl. 514/180; 514/182; 514/462; 514/453; 514/179; 514/177

(57) ABSTRACT

Methods for commercial production of transdermal formulations comprising a hormone compound are provided. In particular, methods for commercial scale production under an inert atmosphere of a transdermal formulation comprising a therapeutically effective amount of a hormone, preferably a testosterone compound, useful for the treatment of hypoactive sexual desire disorder (HSDD) in postmenopausal women are provided.

### CROSS-REFERENCE TO RELATED APPLICATIONS

This application claims the benefit of U.S. Provisional Application No. 61/401,640, filed Aug. 17, 2010, the content of which is hereby incorporated by reference in its entirety.

### FIELD OF THE INVENTION

The present invention relates to improved methods for commercial production of transdermal formulations comprising a hormone. In particular, the present invention relates to methods for commercial scale production under an inert atmosphere of a transdermal formulation comprising a therapeutically effective amount of a hormone, preferably a testosterone compound, useful for the treatment of hypoactive sexual desire disorder (HSDD) in postmenopausal women.

### BACKGROUND

The manufacturing of pharmaceutical compositions on a commercial scale has many technical challenges and hurdles. Often, the small scale methods used to produce the small amount of compound or agent required for laboratory studies and for early clinical testing are not readily amendable to scale up for large-scale production. In some instances, existing methods use processes that may not be technically feasible due to practical limitations or employ operations that are not adaptable or unsafe for large scale production methods (e.g., Bequette, In Pharmaceutical Manufacturing Handbook: production and processes, Vol. 10, Section 3.1, ed. S. Gad, John Wiley & Sons, 2008; Serajuddin J. Pharmacol. Sci 88(10): 1058-1066 (2000)). Successful scale up is critical for pharmaceutical product development and can mean a shortened cycle to full-scale production, competitive advantage, and cost savings (e.g., see Reisman Critical Rev Biotechnol 13(3): (1993)).

The manufacturing process for pharmaceutical compositions typically involves two separate stages. The first stage is the production of the pharmaceutically active agent and the second stage is the formulation process combining the pharmaceutically active agent with a pharmaceutically acceptable carrier suitable for the desired route of administration. Scale up of each of the stages, however, may be hampered at different steps throughout the process (Shah, Comp. Chem Engineer 28:929-941 (2004)).

For instance, certain reactants may be too expensive to purchase in large quantities to cost-effectively produce the compound or active agent at commercial scales. In addition, certain reagents made, for instances, by a single supplier may not even be available in the vast quantities needed for large scale production thereby prohibiting production using the small scale method. The need to produce and formulate the active agent in large volumes creates technical challenges and can introduce increased variability that can decrease product yields across each step of the multistep process thereby increasing the cost of the product obtained from each production run. The midstream development of a new route for the producing the pharmaceutically active agent can be time consuming and resource intensive, and may not yield a viable scaled up method for the product precluding further commercial development.

In the United States, no FDA-approved testosterone therapies are available for the treatment of hypoactive sexual desire disorder (HSDD) in women although in 2009, according to IMS data and independent market research more than 4 million testosterone prescriptions were written “off-label” for women. The unapproved testosterone formulations currently used to treat women have disadvantages. Proper administration, dosing, and daily compliance are a concern when using products for unapproved uses. For example, compounding pharmacies do not follow cGMP processes that are mandated by the FDA for commercial production of pharmaceutical products.

Thus, there remains a significant need to reliably produce formulations comprising a therapeutically effective amount of a hormone at large scale in the volumes required for broad commercial availability. The methods herein desirably accomplish the following attributes at this large scale: 1) complete solubilization and uniform distribution of hormone compound; 2) adequate dispersion of the gelling agent with a subsequent optimum neutralization of the gelling agent; and 3) a final mixing step conducted in a vacuum to prevent entrapped air during gelation and evaporation of the alkanol. The methods herein overcome these issues by providing sufficient quantities of a transdermal gel containing the appropriate amount of a hormone to be used for HSDD, hot flashes and other post-menopausal disorders in a production process according to FDA's cGMP requirements. The resulting gel formulation can be used to deliver transdermally a therapeutically safe and effective amount of a hormone over 24 hours as proven using, in FDA approved clinical trials, the formulation produced by this method.

For example, one goal of the testosterone therapy in postmenopausal women is to increase serum testosterone to the level of a younger woman in an effort to alleviate symptoms of HSDD. Therefore, the resulting formulations produced by the methods of the present invention may be used to treat female sexual dysfunction, particularly HSDD, and the methods are capable of supporting long-term, large scale manufacturing for commercial use.

### SUMMARY OF THE INVENTION

In one aspect of the invention, provided are improved methods for commercial scale production under an inert atmosphere of a transdermal formulation comprising a therapeutically effective amount of a hormone by dissolving a polyalcohol in an amount between 1% and 10% by weight of the formulation and a permeation enhancer in an amount between 1% and 30% by weight of the formulation in an alkanol in an inert atmosphere to form a stirred solution; adding the therapeutically effective amount of the hormone to the stirred solution to form a hormone solution; adding purified water to the hormone solution to form a hydroalcoholic mixture; adding to the hydroalcoholic mixture a sequestering agent in an amount between 0.03% and 0.09% by weight of the formulation to form a second solution; adding a gelling agent to the second solution in an amount between 1% and 10% by weight of the formulation to form a third solution having viscosity between 16,000 and 40,000 cps; adding pH regulator in an amount between 0.2% and 0.5% by weight of the formulation to the third solution to adjust the pH to between 5 and 7 to form the transdermal testosterone formulation; and collecting the transdermal testosterone formulation.

The commercial scale production methods and the formulations of the present invention are suitable for a preparing pharmaceutical compositions comprising a therapeutically effective amount of a variety of hormones. Suitable estrogens for use herein include estrogen and estrogen derivatives such as 17-beta-estradiol and esters thereof, ethinylestradiol, estriol (trihydroxyestrin), estrone, conjugated estrogens, in particular premarin, sodium estrone sulfate, 8(9)-dehydroestradiol derivatives, 17alfa-dihydroequilin, equilenin, 17alfa-dihydroequilenin, esterified estrogens, and equilin. Suitable progesterones for use herein include progesterone, norethisterone acetate, norgestrel, levonorgestrel, gestodene, CPA, chlormadinone acetate, drospirorenone, and 3-ketodesogestrel. Suitable testosterone compounds, including testosterone (17-β-hydroxyandrostenone), testosterone enanthate, testosterone propionate, testosterone decanoate, testosterone cypionate, methyl testosterone, testolactone, oxymetholone, fluoxymesterone and enanthate, propionate, cypionate, phenylacetate, acetate, isobutyrate, buciclate, heptanoate, decanoate, undecanoate, caprate and isocaprate esters of testosterone and 4-dihydrotestosterone.

In certain embodiments, the hormone is an estrogen. The therapeutically effective amount of the estrogen in these transdermal formulation produced by the methods herein is between about 0.01% and 1.00% by weight of the formulation, preferably, between about 0.01% and 0.1% by weight of the formulation, and more preferably the therapeutically effective amount of the estrogen is about 0.06% by weight of the formulation.

In certain embodiments, the hormone is a progesterone. The therapeutically effective amount of the progesterone in these transdermal formulation produced by the methods herein is between about 0.05% and 20.0% by weight of the formulation depending on the progesterone used in the methods.

In certain embodiments, the hormone is a testosterone. The therapeutically effective amount of the testosterone compound in the transdermal formulation produced by the methods herein is between about 0.50% and 2.00% by weight of the formulation, preferably, between about 0.75% and 1.25% by weight of the formulation, and more preferably the therapeutically effective amount of the testosterone compound is about 1.00% by weight of the formulation.

In certain embodiments, the alkanol used is a C₂ to C₄ alcohol such as ethanol, isopropanol, and/or n-propanol, in an amount between about 35 to 55% by weight of the formulation. In preferred embodiments, the alkanol is ethanol an amount of 47.5% by weight of the formulation.

In other embodiments, the alkanol is provided in combination with water to form a hydroalcoholic mixture. Preferably, the alkanol comprises about 45% to 65% and the water comprises about 35% to 55% of the hydroalcoholic mixture by weight. The hydroalcoholic mixture may be present in an amount of about 40 to 98% by weight of the formulation, preferably between 80% and 90% by weight of the formulation.

In certain embodiments, the polyalcohol such as propylene glycol, butylene glycol, hexylene glycol, or ethylene glycol. In certain preferred embodiments, the polyalcohol is propylene glycol.

In certain embodiments, the permeation enhancer is diethylene glycol monomethyl ether, diethylene glycol monoethyl ether or mixtures thereof. In certain preferred embodiments, the permeation enhancer is diethylene glycol monoethyl ether.

The sequestering agent is an edetic acid, preferably edetate disodium.

In certain embodiments, the gelling agent is a polyacrylic acid selected from the group consisting of Carbomer, Carbopol 980 or 940 NF, 981 or 941 NF, 1382 or 1342 NF, and 5984 or 934 NF. In certain preferred embodiments, the gelling agent is Carbomer, Carbopol 980.

In certain embodiments, the pH regulator is triethanolamine, tromethamine, tetrahydroxypropylethylendiamine, or a NaOH solution. In certain preferred embodiments, the pH regulator also contains a cross-linking function and is triethanolamine.

In certain preferred embodiments, the alkanol is a C₂ to C₄ alcohol selected from the group consisting of ethanol, isopropanol, and n-propanol, the polyalcohol is polypropylene glycol, the permeation enhancer is diethylene glycol monomethyl ether, diethylene glycol monoethyl ether, or mixtures thereof, the gelling agent is Carbomer, Carbopol 980, and the pH regulator is triethanolamine.

In other aspects of the invention, the methods include optionally adding a preservative agent, a buffering agent, a moisturizing agent, an emollient, a surfactant, or an antioxidant prior to the step of collecting the transdermal testosterone formulation.

In accordance with a further aspect of the invention, the methods of the present invention further comprise transferring the transdermal testosterone formulation into a metered dosage device to provide convenience as well as precise metered dosages to users. Accordingly, the metered dosage device can be configured to dispense a precise amount of the testosterone compound formulation which corresponds to a desired and prescribed dosage of testosterone compound to the user. Alternatively, the methods of the present invention can further comprise transferring the transdermal testosterone formulation into a unit dose aluminum pouch which is lined with polyethylene, otherwise referred to a sachet or a stick pack.

### DETAILED DESCRIPTION OF THE INVENTION

The present invention relates to methods for commercial scale production under an inert atmosphere of a transdermal formulation comprising a therapeutically effective amount of a hormone by dissolving a polyalcohol in an amount between 1% and 10% by weight of the formulation and a permeation enhancer in an amount between 1% and 30% by weight of the formulation in an alkanol in an inert atmosphere to form a stirred solution; adding the therapeutically effective amount of the hormone to the stirred solution to form a hormone solution; adding purified water to the hormone solution to form a hydroalcoholic mixture; adding to the hydroalcoholic mixture a sequestering agent in an amount between 0.03% and 0.09% by weight of the formulation to form a second solution; adding a gelling agent to the second solution in an amount between 1% and 10% by weight of the formulation to form a third solution having viscosity between 16,000 and 40,000 cps; adding pH regulator in an amount between 0.2% and 0.5% by weight of the formulation to the third solution to adjust the pH to between 5 and 7 to form the transdermal testosterone formulation; and collecting the transdermal testosterone formulation.

### Definitions

Unless defined otherwise, all technical and scientific terms used herein have the same meaning as is commonly understood by one of skill in the art to which this invention belongs. All patents and publications referred to herein are incorporated by reference.

As used herein, “ameliorate” refers to any lessening, whether permanent or temporary, lasting or transient that can be attributed to or associated with administration of the testosterone formulation.

As used herein, a “commercial scale” means that the method for producing a transdermal testosterone formulation is suitable for producing at least 500 kilograms of the transdermal testosterone formulation.

As used herein, “dose” and “dosage” mean a specific amount of a hormone for administration.

As used herein, “estrogen” or “estrogens” has its conventional meaning and comprises estrogen and estrogen derivatives such as 17-beta-estradiol and esters thereof, ethinylestradiol, estriol (trihydroxyestrin), estrone, conjugated estrogens, in particular premarin, sodium estrone sulfate, 8(9)-dehydroestradiol derivatives, 17alfa-dihydroequilin, equilenin, 17alfa-dihydroequilenin, esterified estrogens, and equilin.

As used herein, “hormone” or “hormones” “has its conventional meaning and comprises an estrogen, a progesterone, or a testosterone compound.

As used herein, “progesterone” has its conventional meaning and comprises progesterone, norethisterone acetate, norgestrel, levonorgestrel, gestodene, CPA, chlormadinone acetate, drospirorenone, and 3-ketodesogestrel.

As used herein, “a testosterone compound” means a compound selected from the group consisting of testosterone (17-β-hydroxyandrostenone), testosterone enanthate, testosterone propionate, testosterone decanoate, testosterone cypionate, methyl testosterone, testolactone, oxymetholone, fluoxymesterone and enanthate, propionate, cypionate, phenylacetate, acetate, isobutyrate, buciclate, heptanoate, decanoate, undecanoate, caprate and isocaprate esters of testosterone and 4-dihydrotestosterone.

As used herein, “therapeutically effective amount” means a sufficient amount or dose to provide the desired therapeutic effect.

As used herein, “transdermal formulation” means a formulation for transdermal administration, i.e., delivery by passage of a hormone such as a testosterone through the skin and into the bloodstream.

### Methods for Commercial Scale Production of Transdermal Testosterone Formulations

The methods of the present invention for commercial scale production of a transdermal hormone formulation are suitable for producing batches of at least 500 kilograms of the transdermal hormone formulation. In certain configurations, the instant methods have the capacity to produce greater than 1,000 kilograms or even 1,200 kilograms of the transdermal hormone formulation.

### A. Reaction Vessels

There are a number of reaction vessels suitable for use in the methods herein.

In one embodiment, the manufacturing of transdermal hormone formulations at batch quantities at or exceeding 500 kilograms is performed in two separate reaction vessels. The first vessel is typically a hemispherical, jacketed manufacturing vessel having a capacity of about 700 liters and is used for the preparation of a Primary Compounding Solution. Preferably, this vessel is made of stainless steel.

In one embodiment, the second vessel is a round bottom, jacketed, pressure/vacuum manufacturing vessel having a capacity of about 1350 liters and is used for pH adjustment, final mixing, deaeration, and gel formation. Preferably, this vessel is made of stainless steel, and is capable of maintaining a vacuum of greater than 10 inches of Mercury. A particularly preferred second vessel is a Lee Tri-Mix Turbo Shear manufacturing vessel (Lee Industries, Newton, N.C.).

### B, Preparation of Primary Compounding Solution

The first step in the instant method is dissolving a polyalcohol and a permeation enhancer in an alkanol in an inert atmosphere. The alkanol is placed in a hemispherical, jacketed manufacturing vessel, an inert gas is used to blanket the alkanol in the vessel and the polyalcohol and the permeation enhancer are added to the alkanol while slowly stirring using a dispersion blade within the vessel.

The polyalcohol is added to the alkanol in an amount between 1% and 10% by weight of the formulation and the permeation enhancer is added in an amount between 1% and 30% by weight of the formulation. The inert gas is preferably nitrogen.

The alkanol may be a C₂ to C₄ alcohol, such as ethanol, isopropanol, or n-propanol, and is present in an amount between 35% and 55% by weight of the formulation. In certain preferred embodiments, the alkanol is ethanol. In other preferred embodiment, the ethanol is present in an amount of 47.5%.

In certain embodiments, the polyalcohol may be propylene glycol, butylene glycol, hexylene glycol, and ethylene glycol. In certain preferred embodiments, the polyalcohol is propylene glycol.

The selection of the permeation enhancer can affect the amount and rate of transdermal absorption of hormone formulations. The amount of the permeation enhancer may be optimized. In one preferred embodiment, the permeation enhancer may comprise about 1 to 10% of the formulation by weight. In one embodiment, the permeation enhancer may comprise about 5% of the formulation by weight. In one embodiment, the permeation enhancer is a monoalkyl ether of diethylene glycol. The monoalkyl ether of diethylene is, for example, diethylene glycol monoethyl ether or diethylene glycol monomethyl ether. In certain preferred embodiments, the permeation enhancer is diethylene glycol monoethyl ether. In certain preferred embodiments, the permeation enhancer is diethylene glycol monoethyl ether in an amount of about 5% by weight.

The second step of the present methods is the addition of the therapeutically effective amount of the hormone to the stirred solution to form a testosterone solution. The hormone may advantageously be added as micronized particles and the solution is mixed between 450 and 550 rpm until fully dissolved. Stirring usually occurs for 15 to 30 minutes to allow for the formation of a visually uniform solution lacking any visually detectable particles.

A number of hormones are suitable for use in the methods disclosed herein. Examples of estrogens for use herein include estrogen and estrogen derivatives such as 17-beta-estradiol and esters thereof, ethinylestradiol, estriol (trihydroxyestrin), estrone, conjugated estrogens, in particular premarin, sodium estrone sulfate, 8(9)-dehydroestradiol derivatives, 17alfa-dihydroequilin, equilenin, 17alfa-dihydroequilenin, esterified estrogens, and equilin.

When preparing transdermal estrogen formulations, the therapeutically effective amount of the estrogen in these transdermal formulations is between about 0.01% and 1.00% by weight of the formulation, preferably, between about 0.01% and 0.1% by weight of the formulation, and more preferably the therapeutically effective amount of the estrogen is about 0.06% by weight of the formulation. In preparing the various progesterone formulations, the differences in the amount of progesterone added to the formulation can be balanced by the amount of purified water added on a w/w basis of the formulation.

Examples of progesterones for use herein include progesterone, norethisterone acetate, norgestrel, levonorgestrel, gestodene, CPA, chlormadinone acetate, drospirorenone, and 3-ketodesogestrel. When preparing transdermal progesterone formulations, the progesterones are preferably in amount between: progesterone (10%-20% by weight); norethisterone acetate (1.5%-10% by weight); norgestrel (0.3%-0.8% by weight); levonorgestrel (0.12% -0.4% by weight); gestodene (0.08%-0.15% by weight); CPA (3%-7% by weight); chlormadinone acetate (3%-6% by weight); drospirorenone (3%-10% by weight); and/or 3-ketodesogestrel (0.12%-0.5% by weight). In preparing the various progesterone formulations, the differences in the amount of progesterone added to the formulation can be balanced by the amount of purified water added on a w/w basis of the formulation.

Examples of testosterone compounds which may be used in the present invention include testosterone (17-β-hydroxyandrostenone), and testosterone esters, such as testosterone enanthate, testosterone propionate, testosterone decanoate and testosterone cypionate. The aforementioned testosterone esters are commercially available or may be readily prepared using techniques known to those skilled in the art or described in the pertinent literature. Also, pharmaceutically acceptable esters of testosterone and 4-dihydrotestosterone, typically esters formed from the hydroxyl group present at the C-17 position (such as enanthate, propionate, cypionate, phenylacetate, acetate, isobutyrate, buciclate, heptanoate, decanoate, undecanoate, caprate and isocaprate esters); and pharmaceutically acceptable derivatives of testosterone such as methyl testosterone, testolactone, oxymetholone and fluoxymesterone may be used.

In one embodiment, the therapeutically effective amount of the testosterone compound in the transdermal formulation produced by the methods herein is between about 0.50% and 2.00%, preferably, between about 0.75% and 1.25%, and more preferably the therapeutically effective amount of testosterone is about 1.00%. [by weight?]

The third step of the instant methods is the addition of purified water to form a hydroalcoholic mixture. Preferably, the alkanol comprises about 45% to 65% and the water comprises about 35% to 55% of the hydroalcoholic mixture by weight. The hydroalcoholic mixture may be present in an amount of about 40 to 98% by weight of the formulation, preferably between 80% and 90% by weight of the formulation.

The fourth step of the instant methods is the addition of a sequestering agent. The sequestering agent is present from about 0.03% to about 0.09% w/w of the formulation depending on the type of compound. In preferred embodiments, the sequestering agent is edetic acid. A 3% edetate disodium solution may be prepared, for instances, by dissolving 300 g of edetate disodium USP/EP in 10 kg purified water USP/EP by mixing at about 1,000 rpm for 25 min, and then added to the stirred hydroalcoholic solution to form a visually uniform second solution.

The final step in the preparation of the Primary Compounding Solution is the addition of a gelling agent to the second solution The gelling agent is added to the second solution in an amount sufficient to alter the viscosity of the formulation to result in the desired range of 16,000 to 40,000 cps. The gelling agent can be selected from the group including: carbomer, carboxyethylene or polyacrylic acid such as Carbomer, Carbopol 980 or 940 NF, 981 or 941 NF, 1382 or 1342 NF, 5984 or 934 NF, ETD 2020, 2050, 934P NF, 971P NF, 974P NF, Noveon AA-1 USP; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel different grades), hydroxyethylcellulose (HEC) (Natrosol grades), HPMCP 55, Methocel grades; natural gums such as arabic, xanthan, guar gums, alginates; polyvinylpyrrolidone derivatives such as Kollidon grades; and polyoxyethylene polyoxypropylene copolymers such as Lutrol F grades 68, 127. Other gelling agents may include chitosan, polyvinyl alcohols, pectins, and veegum grades.

In certain preferred embodiments, the gelling agent is one of the Carbomer, Carbopol grade agents, preferably Carbomer, Carbopol 980. The gelling agent may be present from about 0.5% to about 10.0% w/w depending on the type of polymer. In preferred embodiments, the gelling agent is Carbomer, Carbopol 980 present in an amount of 1.2% by weight of the formulation.

### C. Preparation of Transdermal Hormone Formulation

In certain embodiments, the Primary Compounding Solution is transferred into a second round bottom, jacketed, pressure/vacuum vessel for pH adjustment, final mixing, deaeration, and gel formation.

The Primary Compounding Solution placed under vacuum, and mixed prior to the addition of the pH regulator. The pH regulator is generally a neutralizing agent, which can optionally have crosslinking functions. By way of example and not limitation, the pH regulator may include a ternary amine such as triethanolamine, tromethamine, tetrahydroxypropylethylendiamine, or may be a NaOH solution. The pH regulator may be added in an amount between 0.05% to about 2% w/w. In preferred embodiments, the pH regulator is added in an amount between 0.2% to about 0.5% w/w.

In certain preferred embodiments, the pH regulator is triethanolamine (Trolamine). A 17.5% Trolamine solution may be readily prepared by dissolving 1750 g of Trolamine NF in 10 kg purified water USP/EP, and the appropriate amount is calculated and added to the Primary Compounding Solution to reach a final pH between 5.4 and 6.4, which is readily determined by one skilled in the art.

The pH regulator is added to the Primary Compound Solution while stirring at low speeds, e.g., 25 rpm, and then placed under vacuum, e.g., 10-15 inches Hg, for between 90 and 120 minutes to polymerize and deaerate the mixture to form a visually uniform gel yielding about 500 kg of the final hormone transdermal formulation. The final mixing step is conducted in a vacuum to prevent entrapped air during gelation and/or evaporation of the alkanol.

### D. Additional Ingredients

The formulation may further include preservatives such as but not limited to benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives. The preservative may be present from about 0.01 to about 10.0% w/w of the formulation depending on the type of compound.

### Buffering Agent.

The formulation may further include buffers such as carbonate buffers, citrate buffers, phosphate buffers, acetate buffers, hydrochloric acid, lactic acid, tartric acid, diethylamine, triethylamine, triethanolamine, diisopropylamine, aminomethylamine. Other buffers as known in the art may be included additionally or instead. The buffer may replace up to 100% of the water amount within the formulation.

### Moisturizers and Emollients.

Optionally, the formulation may include moisturizers and/or emollients to soften and smooth the skin or to hold and retain moisture. By way of example and not limitation, moisturizers and emollients may include cholesterol, lecithin, light mineral oil, petrolatum, and urea.

### Surfactant.

The formulation may further include anionic, non-ionic or cationic surfactants. The surfactant may be present from about 0.1% to about 30% w/w depending on the type of compound.

### Antioxidants.

The formulation may optionally include antioxidants such as but not limited to tocopherol and derivatives, ascorbic acid and derivatives, butylated hydroxyanisole, butylated hydroxytoluene, fumaric acid, malic acid, propyl gallate, metabisulfates and derivatives. The antioxidant may be present from about 0.001 to about 5.0% w/w of the formulation depending on the type of compound.

For any particular desired formulation, these other ingredients may be selected to achieve the desired drug delivery profile and the amount of penetration desired. The optimum pH may also be determined and may depend on, for example, the base and degree of flux required.

### Methods of Treatment

The formulations produced by the methods of the present invention may be used in methods for treating a postmenopausal woman having HSDD.

In one embodiment, the therapeutically effective amount of testosterone compound is applied directly to the skin The present transdermal therapy provides important advantages over the known oral, intramuscular, and transdermal products by advantageously delivering serum testosterone compound concentrations that are not subject to first-pass metabolism and avoiding wide swings in serum testosterone concentrations while reducing skin reactions often observed with existing transdermal patch products.

The present formulation of the method may be applied once daily, or multiple times per day depending upon the condition of the woman. The formulation may be applied topically to any body part, such as the thigh, abdomen, shoulder, and upper arm. In one embodiment, a formulation in the form of a gel is applied to about a 5 inch by 5 inch area of skin Application may be to alternate areas of the body as applications alternate. For example, the gel may be applied to the thigh for the first application, the upper arm for the second application, and back to the thigh for the third application.

The amount of testosterone compound and dosing schedules necessary to provide a therapeutically effective amount may be monitored by following serum concentrations of testosterone. Methods for measuring the serum levels of such hormones, particularly testosterone, are well known to one of ordinary skill in the art. The serum measures are preferably made when the therapeutically targeted level of steady state has been achieved.

### Metered Dosage Device

A metered dosage device for administration of the transdermal hormone formulation of the present invention may be used in connection with the methods herein. Any metered dosage device capable of dispensing and administering the instant formulations of the methods may be used (e.g., see U.S. Patent Application Publication No. US2006027064).

Preferably, the metered dosage device is capable of dispensing a predetermined, precise amount of a transdermal testosterone formulation produced by the present methods. For example, when used in combination with a gel containing 1% testosterone, the metered dosage device may be designed to dispense 0.22 g of the topical formulation when activated e.g., by pressing on the pump, such that about 2 mg of testosterone is dispensed. Thus, the metered dosage device may be conveniently used for self-administration of a precise testosterone dosage. When smaller doses are to be applied more often, the device can be designed to dispense 1.0 or even 0.5 mg of testosterone upon each activation. Thus, four 0.5 mg doses, two 1 mg doses or a single 2 mg dose can be administered to provide the preferred amount of 2 mg of testosterone each day.

The following Examples are illustrative and are not meant to be limiting.

### EXAMPLE 1

### Commercial-Scale Production of a Transdermal Formulation for Hormones

The following example describes the production of a 500 kg batch of a transdermal formulation according to the methods of the present invention.

Excluding preparation of certain reagents, all procedures throughout the manufacturing process were performed under a nitrogen blanket or vacuum and at ambient temperatures not exceeding 29° C. Solutions were stirred at about 500 rpm unless otherwise noted.

In a pre-charged, round bottom, jacketed, stainless steel 700 liter vessel, 223 kg of ethanol (200 proof) USP/EP was stirred under high shear using a dispersion blade into which 30 kg of propylene glycol USP/EP and 25 kg diethylene glycol monoethyl ether EP/NP were added. The mixture was stirred briefly until visually dissolved and then 173 kg of purified water USP/EP were added using a transfer pump, and the solution was further stirred.

A 3% edetate disodium solution was prepared by dissolving 300 g of edetate disodium USP/EP in 10 kg purified water USP/EP by mixing at about 1,000 rpm for 25 min, and then added to the stirred testosterone solution. After stirring for about 10 min, the speed of mixing was increased to about 650 rpm, and six kilograms of Carbomer, Carbopol 980 dispersion was slowly added to the mixture. The mixture was stirred for 75 minutes yielding a smooth, homogenous solution free of lumps (the Primary Compounding Solution).

The Primary Compounding Solution was transferred into a round bottom, jacketed, stainless steel 1350 liter pressure/vacuum vessel for pH adjustment, final mixing, deaeration, and gel formation. The 700 liter vessel was rinsed with 15 kg of ethanol to ensure complete transfer, added to the Primary Compounding Solution and placed under vacuum. A 17.5% triethanolamine (Trolamine NF) solution was prepared by dissolving 1750 g of Trolamine NF in 10 kg purified water USP/EP. Upon releasing the vacuum, a 7.64 kg amount of Trolamine solution was added to Primary Compounding Solution while stirring at 10 rpm. The mixture was stirred at 25 rpm under vacuum (11 inches of Hg) for 101 minutes to polymerize and deaerate the mixture forming a visually uniform gel yielding about 500 kg of the final transdermal formulation.

The final transdermal formulation consisted of: 47.5% ethanol; 6% propylene glycol; 5% diethylene glycol monoethyl ether; 1.2% Carbomer, Carbopol 980; 0.35% triethanolamine; 0.06% edetate disodium; and 38.89% water. The viscosity of the transdermal gel formulation was 22,000-25,000 cps (Range 16,000-40,000) having a slightly acidic pH, between 5 and 7.

### EXAMPLE 2

### Commercial-Scale Production of a 1% Testosterone Transdermal Formulation

The following example describes the production of a 500 kg batch of a 1% Testosterone transdermal formulation.

Excluding the preparation of certain reagents, all procedures throughout the manufacturing process were performed under a nitrogen blanket or vacuum and at ambient temperatures not exceeding 29° C. Solutions were stirred at about 500 rpm unless otherwise noted.

In a pre-charged, round bottom, jacketed, stainless steel 700 liter vessel, 223 kg of ethanol (200 proof) USP/EP was stirred under high shear using a dispersion blade into which 30 kg of propylene glycol USP/EP and 25 kg diethylene glycol monoethyl ether EP/NP were added. The mixture was stirred briefly until visually dissolved and then five kilograms of testosterone micronized USP/EP were added. After stirring for 17 min, 168 kg of purified water USP/EP were added using a transfer pump, and the solution was further stirred.

A 3% edentate disodium solution was prepared by dissolving 300 g of edetate disodium USP/EP in 10 kg purified water USP/EP by mixing at about 1,000 rpm for 25 min, and then added to the stirred testosterone solution. After stirring for about 10 min, the speed of mixing was increased to about 650 rpm, and six kilograms of Carbomer, Carbopol 980 dispersion was slowly added to the mixture. The mixture was stirred for 75 minutes yielding a smooth, homogenous solution free of lumps (the Primary Compounding Solution).

The Primary Compounding Solution was transferred into a round bottom, jacketed, stainless steel 1350 liter pressure/vacuum vessel for pH adjustment, final mixing, deaeration, and gel formation. The 700 liter vessel was rinsed with 15 kg of ethanol to ensure complete transfer, added to the Primary Compounding Solution and placed under vacuum. A 17.5% triethanolamine (Trolamine NF) solution was prepared by dissolving 1750 g of Trolamine NF in 10 kg purified water USP/EP. Upon releasing the vacuum, a 7.64 kg amount of Trolamine solution was added to Primary Compounding Solution while stirring at 10 rpm. The mixture was stirred at 25 rpm under vacuum (11 inches of Hg) for 101 minutes to polymerize and deaerate the mixture forming a visually uniform gel yielding about 500 kg of the final testosterone transdermal formulation.

The final transdermal testosterone formulation consisted of: 1% testosterone; 47.5% ethanol; 6% propylene glycol; 5% diethylene glycol monoethyl ether; 1.2% Carbomer, Carbopol 980; 0.35% triethanolamine; 0.06% edetate disodium; and 38.89% water. The viscosity of the transdermal gel formulation was 22,000-25,000 cps (Range 16,000-40,000) having a slightly acidic pH, between 5 and 7.

What is claimed is:

1. A method for commercial scale production under an inert atmosphere of a transdermal formulation comprising a therapeutically effective amount of a hormone, the method comprising the steps of:
dissolving a polyalcohol in an amount between 1% and 10% by weight of the formulation and a permeation enhancer in an amount between 1% and 30% by weight of the formulation in an alkanol in an inert atmosphere to form a stirred solution;
adding the therapeutically effective amount of the hormone to the stirred solution to form a hormone solution;
adding purified water to the hormone solution to form a hydroalcoholic mixture;
adding to the hydroalcoholic mixture a sequestering agent in an amount between 0.03% and 0.09% by weight of the formulation to form a second solution;
adding a gelling agent to the second solution in an amount between 0.5% and 10% by weight of the formulation to form a primary compounding solution having viscosity between 16,000 and 40,000 cps; and
adding a pH regulator in an amount between 0.2% and 0.5% by weight of the formulation to the primary compounding solution to adjust the pH to between 5 and 7 to form the transdermal hormone formulation.

2. The method according to claim 1, further comprising collecting the transdermal hormone formulation.

3. The method according to claim 1, wherein the hormone is an estrogen selected from the group consisting of estrogen, 17-beta-estradiol and esters thereof, ethinylestradiol, estriol (trihydroxyestrin), estrone, conjugated estrogens, in particular premarin, sodium estrone sulfate, 8(9)-dehydroestradiol derivatives, 17alfa-dihydroequilin, equilenin, 17alfa-dihydroequilenin, esterified estrogens, and equilin.

4. The method according to claim 1, wherein the hormone is a progesterone selected from the group consisting of progesterone, norethisterone acetate, norgestrel, levonorgestrel, gestodene, CPA, chlormadinone acetate, drospirorenone, and 3-ketodesogestrel.

5. The method according to claim 1, wherein the hormone is a testosterone compound selected from the group consisting of testosterone (17-β-hydroxyandrostenone), testosterone enanthate, testosterone propionate, testosterone decanoate, testosterone cypionate, methyl testosterone, testolactone, oxymetholone, fluoxymesterone and enanthate, propionate, cypionate, phenylacetate, acetate, isobutyrate, buciclate, heptanoate, decanoate, undecanoate, caprate and isocaprate esters of testosterone and 4-dihydrotestosterone.

6. The method according to claim 5, wherein the therapeutically effective amount of the testosterone compound is between about 0.50% and 2.00% by weight of the formulation.

7. The method according to claim 6, wherein the therapeutically effective amount of the testosterone compound is between about 0.75% and 1.25% by weight of the formulation.

8. The method according to claim 7, wherein the therapeutically effective amount of the testosterone compound is about 1.00% by weight of the formulation.

9. The method accordingly to claim 8, wherein the testosterone compound is testosterone.

10. The method according to claim 1, wherein the alkanol is a C₂ to C₄ alcohol selected from the group consisting of ethanol, isopropanol, and n-propanol, and is present in an amount between 35% and 55% by weight of the formulation.

11. The method according to claim 1, wherein the polyalcohol is propylene glycol, butylene glycol, hexylene glycol, or ethylene glycol,

12. The method according to claim 1, wherein the permeation enhancer is diethylene glycol monoethyl ether, diethylene glycol monomethyl ether, or mixtures thereof.

13. The method according to claim 1, wherein the sequestering agent is an edetic acid in the form of edentate disodium.

14. The method according to claim 1, wherein the gelling agent is a polyacrylic acid Carbomer selected from the group consisting of Carbomer, Carbopol 980 or 940 NF, 981 or 941 NF, 1382 or 1342 NF, and 5984 or 934 NF.

15. The method according to claim 1, wherein the pH regulator is triethanolamine, tromethamine, tetrahydroxypropylethylendiamine, or a NaOH solution.

16. The method according to claim 9, wherein the alkanol is a C₂ to C₄ alcohol selected from the group consisting of ethanol, isopropanol, and n-propanol, the polyalcohol is polypropylene glycol, the permeation enhancer is diethylene glycol monomethyl ether, diethylene glycol monoethyl ether, or mixtures thereof, the gelling agent is Carbomer, Carbopol 980, and the pH regulator is triethanolamine.

17. The method according to claim 15, which further comprises transferring the collected transdermal testeosterone formulation to a holding vessel.

18. The method according to claim 15, which further comprises transferring the harvested transdermal testeosterone formulation to a metered dosage device.

19. The method according to claim 18, wherein the metered dosage device accurately controls the administration of testosterone compound by dispensing a precise amount of testosterone for self administration upon a transdermal surface of the subject.

20. A method for producing under an inert atmosphere a 500 kilogram batch of a transdermal formulation comprising a therapeutically effective amount of a testosterone compound, the method comprising the steps of:
dissolving 5.0 kilograms of a testosterone compound, 25 kilograms of propylene glycol and 30 kilograms diethylene glycol monoethyl ether in 223 kg of stirred 200 proof ethanol to form a stirred solution;
adding 168 kg purified water to the stirred solution to form a hydroalcoholic mixture;
adding to the hydroalcoholic mixture while stirring 300 grams of edetate disodium dissolved in 10 kg purified water to form a second solution;
adding 6.0 kilograms of Carbomer Carbopol 980 to the second solution to form a third solution having viscosity between 22,000 and 25,000 cps;
adding 7.64 kilograms of triethanolamine to the third solution to adjust the pH to between 5 and 7 to form the transdermal testosterone formulation; and
collecting about 500 kilograms of the transdermal testosterone formulation.
